SEQUENCE LISTING (79 sequences)

SEQ ID NO: 1 - protein, 16 aa, synthetic construct
VVSVLTVLHQDWQNGK

SEQ ID NO: 2 - protein, 16 aa, synthetic construct
VVSVLTVLHQDWLNGK

SEQ ID NO: 3 - protein, 10 aa, synthetic construct
NQASLTCLVK

SEQ ID NO: 4 - protein, 10 aa, synthetic construct
NQVSLTCLVK

SEQ ID NO: 5 - protein, 14 aa, synthetic construct
DSTYNLSSTLTLSK

SEQ ID NO: 6 - protein, 14 aa, synthetic construct
DSTYSLSSTLTLSK

SEQ ID NO: 7 - protein, 12 aa, synthetic construct
GPSJFPLAPSSK

SEQ ID NO: 8 - protein, 16 aa, synthetic construct
VVNVLTVLHQDWLNGK

SEQ ID NO: 9 - protein, 17 aa, synthetic construct
TTPPVLDSDGSFFLYNK

SEQ ID NO: 10 - protein, 17 aa, synthetic construct
VYACEVTHQDLSSPVTK

SEQ ID NO: 11 - protein, 12 aa, synthetic construct
GPSVFPLAPSTK

SEQ ID NO: 12 - protein, 22 aa, synthetic construct
GFYPSDIAVEWESNGQPESNYK

SEQ ID NO: 13 - protein, 17 aa, synthetic construct
TTPPVLDSDGSJFLYSR

SEQ ID NO: 14 - protein, 14 aa, synthetic construct
TYTYNVDHKPSNTK

SEQ ID NO: 15 - protein, 17 aa, synthetic construct
VYAYEVTHQGLSSPVTK

SEQ ID NO: 16 - protein, 330 aa, synthetic construct [sequence omitted]

SEQ ID NO: 17 - protein, 330 aa, synthetic construct
  VARIANT 8: note = X can be any amino acid
ASTKGPSXFPLAPSTKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVNVLTVLHQDWQNGKEYKCKVSNKALPAPIEKTISKAKGQPREPQVYTLPPSRDELTKNQASLTCLVKGFYPSDIAVEWESNGQPESNYKTTPPVLDSDGSFFLYNKLTVDKSRWQQGSVFSCSVMHEALHNHYTQKSLSLSPGK

SEQ ID NO: 18 - protein, 214 aa, synthetic construct
DIQMTQSPSTLSASVGDRVTITCRASQSINTWLAWYQQKPGKAPKLLMYKASSLESGVPSRFIGSGSGTEFTLTISSLQPDDFATYYCQQYNSDSKMFGQGTKVEVKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 214 aa, synthetic construct
DIQMTQSPSTLSASVGDRVTITCRASQSINTWLAWYQQKPGKAPKLLMYKASSLESGVPSRFIGSGSGTEFTLTISSLQPDDFATYYCQQYNSDSKMFGQGTKVEVKGTVAAPSVFIFPPSDEQLKSGTASVVCLLNNFYPREAKVQWKVDNALQSGNSQESVTEQDSKDSTYNLSSTLTLSKADYEKHKVYAYEVTHQDLSSPVTKSFNRGEC

SEQ ID NO: 20 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, synthetic construct
  VARIANT 284: note = X can be any amino acid
ASTKGPSVFPLAPCSRSTSESTAALGCLVKDYFPEPVTVSWNSGALTSGVHTFPAVLQSSGLYSLSSVVTVPSSSLGTKTYTYNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVNVLTVLHQDWLNGKEYKCKVSNKGLPSSIEKTISKAKGQPREPQVYTLPPSQEEMTKNQASLTCLVKGFYPSDIAVEWESNGQPENNYKTTPPVLDSDGSXFLYSRLTVDKSRWQEGNVFSCSVMHEALHNHYTQKSLSLSLGK

SEQ ID NO: 22 - protein, 22 aa, synthetic construct
GFYPSDIAVEWESNGQPENNYK

SEQ ID NO: 23 - protein, 12 aa, synthetic construct
GPSVFPLAPSSK

SEQ ID NO: 24 - protein, 12 aa, synthetic construct
GPSLFPLAPSSK

SEQ ID NO: 25 - protein, 12 aa, synthetic construct
  VARIANT 4: note = X can be any amino acid
GPSXFPLAPSSK

SEQ ID NO: 26 - protein, 12 aa, synthetic construct
GPSIFPLAPSSK

SEQ ID NO: 27 - protein, 12 aa, synthetic construct
SGGTAALGCLVK

SEQ ID NO: 28 - protein, 17 aa, synthetic construct
TTPPVLDSDGSFLLYSR

SEQ ID NO: 29 - protein, 14 aa, synthetic construct
DSTYSLSSTLTLNK

SEQ ID NO: 30 - protein, 19 aa, synthetic construct
TPEVTCVVVDVNHEDPEVK

SEQ ID NO: 31 - protein, 14 aa, synthetic construct
FNWYVDDVEVHNAK

SEQ ID NO: 32 - protein, 14 aa, synthetic construct
STSGDTAALGCLVK

SEQ ID NO: 33 - protein, 12 aa, synthetic construct
DPSVFPLAPSSK

SEQ ID NO: 34 - protein, 8 aa, synthetic construct
  VARIANT 5: note = X can be any amino acid
LASGXPSR

SEQ ID NO: 35 - protein, 16 aa, synthetic construct
  VARIANT 7: note = X can be any amino acid
VVSVLTXLHQDWLNGK

SEQ ID NO: 36 - protein, 16 aa, synthetic construct
  VARIANT 4: note = X can be any amino acid
VVSXLTVLHQDWLNGK

SEQ ID NO: 37 - protein, 12 aa, synthetic construct
GPSVFPLTPSSK

SEQ ID NO: 38 - protein, 14 aa, synthetic construct
STSGGTAALGCLVR

SEQ ID NO: 39 - protein, 8 aa, synthetic construct
LASGVPSR

SEQ ID NO: 40 - protein, 10 aa, synthetic construct
VTITCSASSR

SEQ ID NO: 41 - protein, 8 aa, synthetic construct
ALPAPIEK

SEQ ID NO: 42 - protein, 17 aa, synthetic construct
VYACEVTHQGLSSPVTK

SEQ ID NO: 43 - protein, 7 aa, synthetic construct
SLSLSPG

SEQ ID NO: 44 - protein, 8 aa, synthetic construct
LLIYDTSK

SEQ ID NO: 45 - protein, 14 aa, synthetic construct
STSGGTAALGCLVK

SEQ ID NO: 46 - protein, 23 aa, synthetic construct
WQQGNVFSCSVMHEALHNHYTQK

SEQ ID NO: 47 - protein, 14 aa, synthetic construct
FNWYVDGVEVHNAK

SEQ ID NO: 48 - protein, 19 aa, synthetic construct
TPEVTCVVVDVSHEDPEVK

SEQ ID NO: 49 - protein, 17 aa, synthetic construct
TTPPVLDSDGSFFLYSK

SEQ ID NO: 50 - protein, 63 aa, synthetic construct
DYFPEPVTVSWNSGALTSGVHTFPAVLQSSGLYSLSSVVTVPSSSLGTQTYICNVNHKPSNTK

SEQ ID NO: 51 - protein, 16 aa, synthetic construct
KDSTYSLSSTLTLNKA

SEQ ID NO: 52 - protein, 16 aa, synthetic construct
KFNWYVDGMEVHNAKT

SEQ ID NO: 53 - protein, 16 aa, synthetic construct
KFNWYVDDVEVHNAKT

SEQ ID NO: 54 - protein, 16 aa, synthetic construct
KFNWYMDGVEVHNAKT

SEQ ID NO: 55 - protein, 16 aa, synthetic construct
KNTSGGTAALGCLVKD

SEQ ID NO: 56 - protein, 14 aa, synthetic construct
KGPSVFPLTPSSKS

SEQ ID NO: 57 - protein, 14 aa, synthetic construct
  VARIANT 5: note = X can be any amino acid
KGPSXFPLAPSSKS

SEQ ID NO: 58 - protein, 14 aa, synthetic construct
  VARIANT 4: note = X can be any amino acid
KGPXVFPLAPSSKS

SEQ ID NO: 59 - protein, 10 aa, synthetic construct
  VARIANT 6: note = X can be any amino acid
KLASGXPSRF

SEQ ID NO: 60 - protein, 10 aa, synthetic construct
KLLIYDISKL

SEQ ID NO: 61 - protein, 12 aa, synthetic construct
  VARIANT 4: note = X can be any amino acid
KNQXSLTCLVKG

SEQ ID NO: 62 - protein, 16 aa, synthetic construct
KSTSGGTAALGCLVRD

SEQ ID NO: 63 - protein, 16 aa, synthetic construct
KSTSGGTAALDCLVKD

SEQ ID NO: 64 - protein, 16 aa, synthetic construct
KSTSGGTATLGCLVKD

SEQ ID NO: 65 - protein, 16 aa, synthetic construct
KSTSGDTAALGCLVKD

SEQ ID NO: 66 - protein, 19 aa, synthetic construct
KTTPPVLDSDGSFFLYNKL

SEQ ID NO: 67 - protein, 19 aa, synthetic construct
KVYACEVTHQDLSSPVTKS

SEQ ID NO: 68 - protein, 24 aa, synthetic construct
KGFYPSDIAVEWENNGQPENNYKT

SEQ ID NO: 69 - protein, 14 aa, synthetic construct
KDPSVFPLAPSSKS

SEQ ID NO: 70 - protein, 9 aa, synthetic construct
KNLSLSPGK

SEQ ID NO: 71 - protein, 19 aa, synthetic construct
  VARIANT 2: note = X can be any amino acid
KXYACEVTHQGLSSPVTKS

SEQ ID NO: 72 - protein, 21 aa, synthetic construct
RTPEVTCVVVDVNHEDPEVKF

SEQ ID NO: 73 - protein, 23 aa, synthetic construct
  VARIANT 12: note = X can be V or R
RVTITCSASNRXVTITCNASSRV

SEQ ID NO: 74 - protein, 16 aa, synthetic construct
RVVSVLTVLHQDWLKG

SEQ ID NO: 75 - protein, 18 aa, synthetic construct
  VARIANT 5: note = X can be any amino acid
RVVSXLTVLHQDWLNGKE

SEQ ID NO: 76 - protein, 18 aa, synthetic construct
RVVNVLTVLHQDWLNGKE

SEQ ID NO: 77 - protein, 10 aa, synthetic construct
KLASGVPSKF

SEQ ID NO: 78 - protein, 25 aa, synthetic construct
  VARIANT 13: note = X can be any amino acid
RWQQGNVFSCSVXHEALHNHYTQKS

SEQ ID NO: 79 - protein, 10 aa, synthetic construct
KTLPAPIEKT

(19) United States
(12) Patent Application Publication
(10) Pub. No.: US 2024/0142462 A1
(43) Pub. Date: May 2, 2024

(54) SEQUENCE VARIANT ANALYSIS USING HEAVY PEPTIDES

(71) Applicant: Regeneron Pharmaceuticals, Inc., Tarrytown, NY (US)

(72) Inventors: Tyler Greer, Elmsford, NY (US); Reid O'Brien-Johnson, Hartsdale, NY (US); Milos Cejkov, Ridgewood, NJ (US); Xiaojing Zheng, Croton-on-Hudson, NY (US); Ning Li, New Canaan, CT (US)

(21) Appl. No.: 18/110,130

(22) Filed: Feb. 15, 2023

Related U.S. Application Data
(60) Provisional application No. 63/310,626, filed on Feb. 16, 2022.

(51) Int. Cl. G01N 33/68; G01N 30/02; G01N 30/72; G01N 30/86

(52) U.S. Cl. CPC G01N 33/6848; G01N 30/7266; G01N 30/8631; G01N 2030/027

(57) ABSTRACT

The present invention generally pertains to methods of identifying sequence variants in a protein of interest. In particular, the present invention pertains to the use of heavy peptide standards with liquid chromatography-mass spectrometry analysis to specifically identify sequence variants of a protein of interest.

### CROSS-REFERENCE TO RELATED APPLICATIONS

This application claims priority to and the benefit of U.S. Provisional Patent Application No. 63/310,626 filed Feb. 16, 2022.

### SEQUENCE LISTING

This instant application contains a Sequence Listing which has been submitted electronically in XML format and is hereby incorporated by reference in its entirety. Said XML copy, created on Jun. 16, 2023, is named 070816-02981 SL.xml and is 76,980 bytes in size.

### FIELD

This application relates to methods for identification of sequence variants in a protein of interest.

### BACKGROUND

Characterization of therapeutic antibodies' critical quality attributes (CQAs) is important due to the large size and complex heterogeneity of this major class of therapeutics. One such CQA is any variation in the amino acid sequence of the protein, called sequence variants (SVs). The presence of elevated SVs contributes to product heterogeneity and could also affect drug efficacy and safety if, for example, the amino acid variants are located in binding regions, or introduce a non-human amino acid sequence.

Detecting sequence variants presents a challenge because of their typically low abundance. Sequence variants may exist at, for example, 0.001% to 0.1% the abundance of the corresponding non-variant peptides. High quality MS² spectra with nearly complete backbone fragmentation are required to properly identify and isolate the variant amino acid. In the absence of entirely unambiguous and clear spectral data, false negative or false positive identifications of sequence variants are possible

Therefore, demand exists for methods and systems to identify sequence variants in antibody products in a sensitive and specific fashion.

### SUMMARY

A method has been developed for confidently identifying an amino acid sequence of a peptide of a protein of interest, for example to identify sequence variants in an antibody product. The method includes the novel use of heavy peptide standards comprising a heavy isotope at or near each peptide terminus. Heavy peptide standards may be selected that comprise an amino acid sequence corresponding to a predicted amino acid sequence of a digested peptide of a protein of interest. The predicted amino acid sequence may be, for example, the wildtype amino acid sequence, a mutant amino acid sequence, or a sequence variant. Using liquid chromatography, the retention time of a heavy peptide standard will align with the retention time of a corresponding digested peptide, which controls for any experimental variation in retention time. Using mass spectrometry, MS¹ peaks from a heavy peptide standard will be shifted from a corresponding digested peptide by the mass of the heavy isotopes, allowing for clear confirmation of the digested peptide mass. MS² peaks from a heavy peptide standard will be shifted from a corresponding digested peptide by the mass of the heavy isotope at or near the included peptide terminus, allowing for clear identification of each amino acid of the digested peptide. Using the method of the present invention, it can be confidently determined whether a digested peptide features a predicted amino acid sequence, for example a wildtype amino acid sequence, a mutant amino acid sequence or a sequence variant. Conversely, a false positive identification of an amino acid sequence may be refuted by comparison with a heavy peptide standard known to feature the amino acid sequence.

This disclosure provides a method for identifying an amino acid sequence of a digested peptide of a protein of interest. In some exemplary embodiments, the method comprises (a) combining a peptide digest having digested peptides of a protein of interest with at least one heavy peptide standard to form a mixture, wherein said at least one heavy peptide standard includes a heavy isotope at or near each peptide terminus and an amino acid sequence of said at least one heavy peptide standard is a predicted amino acid sequence of a digested peptide of said protein of interest; (b) subjecting said mixture to analysis using liquid chromatography-mass spectrometry; (c) comparing a retention time and/or at least one mass spectrum of said at least one heavy peptide standard to a retention time and/or at least one mass spectrum of said digested peptides; and (d) using the comparison of (c) to identify the amino acid sequence of a digested peptide of said protein of interest.

In one aspect, the amino acid sequence is a sequence variant. In a specific aspect, the sequence variant is a critical quality attribute.

In one aspect, the protein of interest can be an antibody, a bispecific antibody, a monoclonal antibody, a fusion protein, an antibody-drug conjugate, an antibody fragment, a host cell protein, or a protein pharmaceutical product.

In one aspect, the chromatography step comprises reversed phase liquid chromatography, ion exchange chromatography, size exclusion chromatography, affinity chromatography, hydrophobic interaction chromatography, hydrophilic interaction chromatography, mixed-mode chromatography, or a combination thereof.

In one aspect, the mass spectrometer is an electrospray ionization mass spectrometer, nano-electrospray ionization mass spectrometer, or an Orbitrap-based mass spectrometer, wherein said mass spectrometer is coupled to the liquid chromatography system.

In one aspect, said at least one mass spectrum is an MS¹ spectrum. In another aspect, said at least one mass spectrum is an MS² spectrum (tandem mass spectrum). In a further aspect, said at least one mass spectrum is an MS³ spectrum.

In one aspect, the comparing step comprises determining whether the retention time of the at least one heavy peptide standard aligns with a retention time of a digested peptide. In another aspect, the comparing step comprises determining whether the MS¹ spectrum peaks of the at least one heavy peptide are shifted by the added mass of the heavy isotopes relative to a digested peptide. In a further aspect, the comparing step comprises determining whether the MS² spectrum peaks of the at least one heavy peptide are shifted by the added mass of one of the heavy isotopes relative to a digested peptide.

In one aspect, the comparing step further comprises comparing at least one chromatogram of the at least one heavy peptide standard to at least one chromatogram of a digested peptide. In a specific aspect, the comparing step comprises determining whether the main peak of the at least one heavy peptide standard substantially overlaps with the main peak of a digested peptide. In a further specific aspect, the comparing step comprises determining whether the main peak of the at least one heavy peptide completely overlaps with the main peak of a digested peptide.

In one aspect, a molar ratio of the peptide digest to the heavy peptide standard is between about 1:50 and 1:200, about 1:100, or 1:100.

These, and other, aspects of the present invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows mass differences between post-translational modifications and similar sequence variants, according to an exemplary embodiment.

FIG. 2A (SEQ ID NO:1 AND 2) shows mass spectra of a previously identified sequence variant that was shown to be a false positive using the method of the present invention, according to an exemplary embodiment.

FIG. 2B (SEQ ID NO:3 and 4) shows mass spectra of a previously identified sequence variant that was shown to be a false positive using the method of the present invention, according to an exemplary embodiment.

FIG. 2C (SEQ ID NO:5 and 6) shows mass spectra of a previously identified sequence variant that was shown to be a true positive using the method of the present invention, according to an exemplary embodiment.

FIG. 3 illustrates a workflow of the method of the present invention, according to an exemplary embodiment.

FIG. 4 illustrates a comparison of a heavy peptide standard to a digested peptide using liquid chromatography retention time, MS¹ spectra and MS² spectra, according to an exemplary embodiment.

FIG. 5(SEQ ID NO:7-15) shows amino acid sequences of heavy peptide standards, according to an exemplary embodiment.

FIG. 6(SEQ ID NO:16-21) shows regions of an antibody selected for analysis using heavy peptide standards, according to an exemplary embodiment.

FIG. 7(SEQ ID NO:22) shows a liquid chromatography and MS¹ analysis with no sequence variants identified, according to an exemplary embodiment.

FIG. 8A shows a liquid chromatography and MS¹ analysis with a sequence variant identified, according to an exemplary embodiment. FIG. 8B shows a MS² analysis with an amino acid sequence of a sequence variant identified, according to an exemplary embodiment.

FIG. 9A (SEQ ID NO:23-25) shows a liquid chromatography and MS¹ analysis with an undetermined sequence variant identified, according to an exemplary embodiment. FIG. 9B shows an MS² analysis with an undetermined leucine or isoleucine sequence variant identified, according to an exemplary embodiment. FIG. 9C (SEQ ID NO:26) shows a difference in retention times of heavy peptide standards corresponding to a leucine or an isoleucine sequence variant, according to an exemplary embodiment. FIG. 9D shows a liquid chromatography analysis with a sequence variant identified as isoleucine, according to an exemplary embodiment.

FIG. 10A shows a liquid chromatography and MS¹ analysis with a putative sequence variant, according to an exemplary embodiment. FIG. 10B shows a MS² analysis with a putative sequence variant featuring an unconfirmed amino acid sequence, according to an exemplary embodiment. FIG. 10C shows a liquid chromatography and MS¹ analysis refuting the false positive identification of a putative sequence variant, according to an exemplary embodiment. FIG. 10D shows a liquid chromatography and MS² analysis refuting a false positive identification of a putative sequence variant, according to an exemplary embodiment.

FIG. 10E (SEQ ID NO:27) shows a liquid chromatography and MS² analysis identifying a putative sequence variant as a non-specific cleavage product, according to an exemplary embodiment.

FIG. 11 (SEQ ID NO:28) shows previously identified sequence variants that were confirmed or refuted using the method of the present invention, according to an exemplary embodiment.

FIG. 12(SEQ ID NO:29-38) shows known NISTmAb sequence variants used to benchmark the liquid chromatography step of the method of the present invention, according to an exemplary embodiment.

FIG. 13 (SEQ ID NO:39-50) shows standard deviations of retention times of digested peptides across five tested gradient durations, according to an exemplary embodiment.

FIG. 14 (SEQ ID NO:51-79) shows total sequence variants identified across five tested gradient durations, according to an exemplary embodiment.

FIG. 15 shows validation of previously identified sequence variants across four tested gradient durations, according to an exemplary embodiment.

FIG. 16 shows Byonic scores for MS² spectra of sequence variants identified across five tested gradient durations, according to an exemplary embodiment.

FIG. 17 shows quantitative signal of sequence variants identified across five tested gradient durations, according to an exemplary embodiment.

### DETAILED DESCRIPTION

Characterization of therapeutic antibodies' critical quality attributes (CQAs) is important due to the large size and complex heterogeneity of this increasingly popular class of therapeutics. One such CQA is sequence variants caused by substitution of an amino acid. Sequence variants can be caused, for example, by DNA mutation to the production cell line, or by translational errors during protein production. Elevated levels of sequence variants contribute to product heterogeneity and may affect efficacy or safety if amino acid variants are located in binding regions or introduce a non-human amino acid sequence.

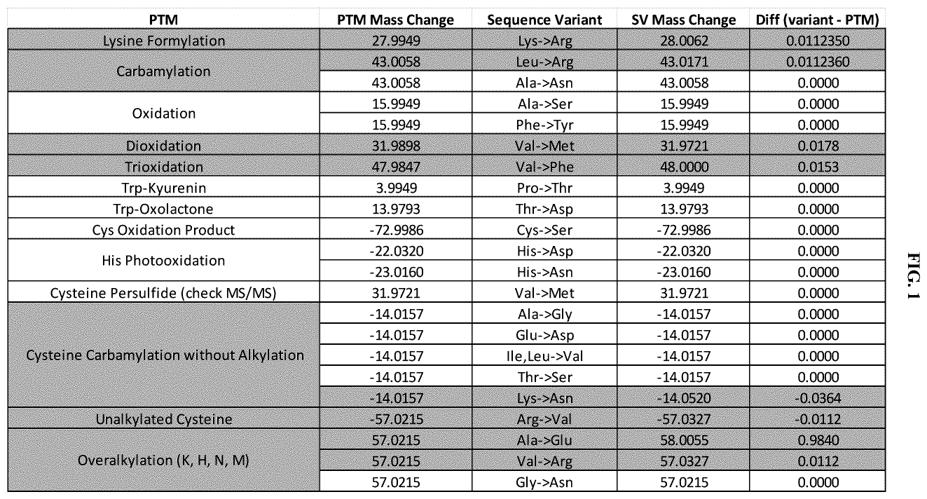

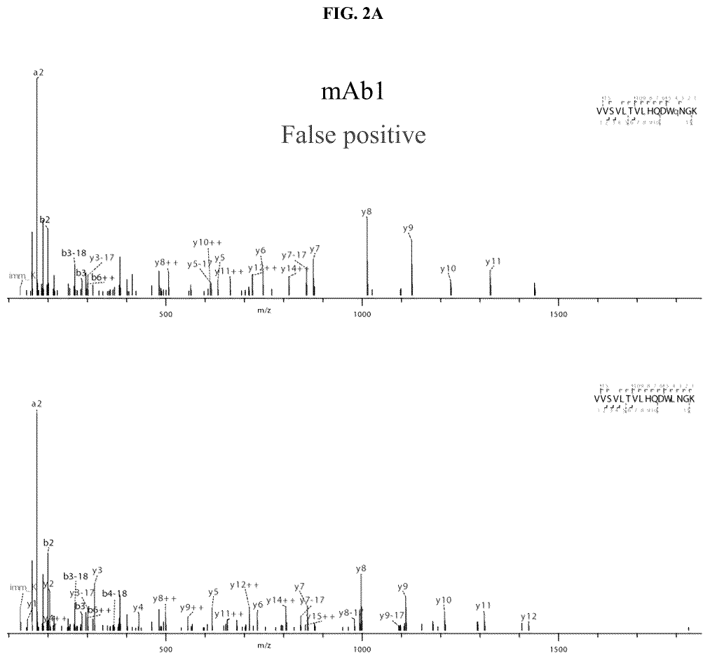

Detecting sequence variants presents a challenge because of their typically low abundance. Sequence variants may exist at, for example, 0.001% to 0.1% the abundance of the corresponding non-variant peptides. High quality MS² spectra with nearly complete backbone fragmentation are required to properly identify and isolate the variant amino acid. The difficulty of distinguishing sequence variants from other CQAs, for example non-specific cleavages or post-translational modifications (PTMs), is illustrated in FIG. 1. The mass difference between PTMs and substituted amino acids may be in the sub-ppm (parts per million) range, which requires the use of equipment with particularly sensitive detection. In FIG. 1, green rows indicate PTM masses that are very close to sequence variant masses, and white rows indicate PTM masses that are identical to sequence variant masses and cannot be differentiated using mass spectrometry.

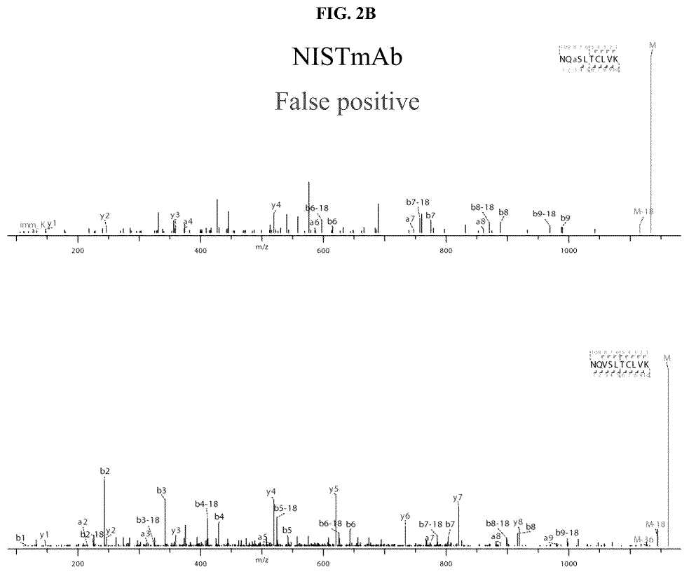

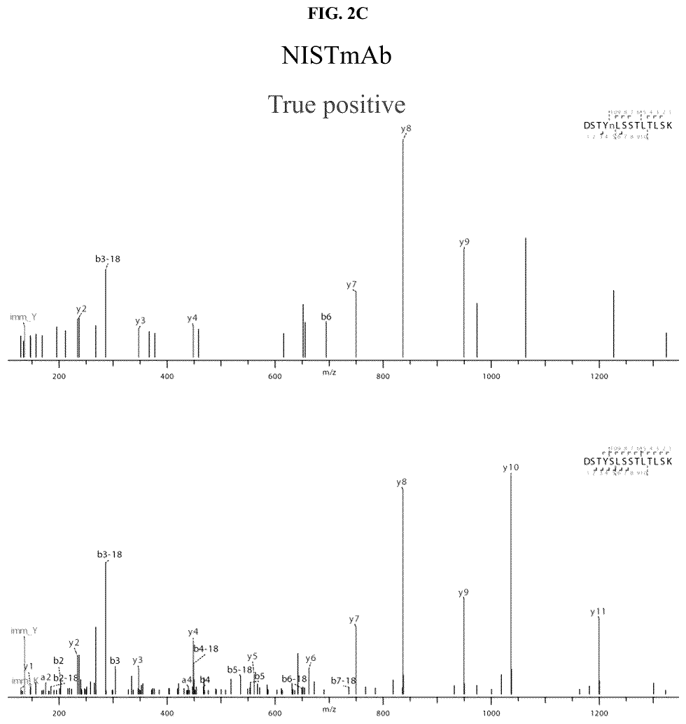

Confidence in identification of sequence variants is further decreased when MS² spectra are ambiguous or misleading. Examples of peptides that were identified as sequence variants based on their mass spectra are shown in FIG. 2A, FIG. 2B, and FIG. 2C. Two of the examples show false positive identifications, caused by a confounding non-specific cleavage in one case (FIG. 2A) and a confounding combination of PTMs in another case (FIG. 2B), illustrating that distinguishing false positive from true positive sequence variant identifications can be difficult when performed without a standard of comparison.

Confidence in sequence variant identification is higher when multiple sequence variants exist. In contrast, there is less confidence in the accuracy of a sequence variant identification when only one variant is identified.

The disclosure herein provides a solution to confirming true positive identifications of sequence variants and ruling out false positive identifications of sequence variants in a protein of interest. A heavy peptide standard comprising heavy isotopes at or near both peptide termini provides a standard of comparison against putative sequence variant peptides that will have an overlapping retention time in a liquid chromatography system but be clearly separable and comparable in mass spectra. The method of the present invention may be used, for example, to assess CQAs in a therapeutic antibody, including, for example, sequence variants. The method may further be used to confirm peptide sequence and identity in the analysis of any protein of interest.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration.

In some exemplary embodiments, a sample including a protein of interest can be prepared prior to LC-MS analysis. Preparation steps can include denaturation, alkylation, dilution and digestion.

As used herein, the term “protein alkylating agent” or “alkylation agent” refers to an agent used for alkylating certain free amino acid residues in a protein. Non-limiting examples of protein alkylating agents are iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

As used herein, the term “protein reducing agent” or “reduction agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds.

In some exemplary embodiments, a heavy peptide standard may be added to a peptide digest. As used herein, the term “heavy peptide standard” refers to a peptide with a known amino acid sequence that comprises at least one heavy isotope. A heavy peptide standard may be compared to a peptide of unknown amino acid sequence in order to identify the unknown amino acid sequence. For example, a heavy peptide standard may be compared to another peptide on the basis of retention time using liquid chromatography or mass spectral signal using a mass spectrometer. A heavy peptide standard may be expected to have a chromatographic peak that substantially overlaps with a chromatographic peak from another peptide with an identical amino acid sequence, which may also be referred to as aligned retention times.

Heavy peptide standards that are particularly useful in the method of the present invention include heavy peptide standards comprising a heavy isotope at or near each peptide terminus. A heavy isotope near a peptide terminus may be, for example, one amino acid away from the terminus or two amino acids away from the terminus. The inclusion of a heavy isotope at or near each terminus ensures that every or nearly every fragment ion in a tandem mass spectrum, which include either the N-terminus or the C-terminus of a fragmented peptide, will be shifted by the mass of the corresponding heavy isotope, allowing for differentiation from and comparison to another peptide of the same amino acid sequence in an MS² analysis.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase liquid chromatography, ion-exchange chromatography, size exclusion chromatography, affinity chromatography, hydrophobic interaction chromatography, hydrophilic interaction chromatography, or mixed-mode chromatography. In some aspects, the sample containing the at least one protein of interest or peptide digest can be subjected to any one of the aforementioned chromatographic methods or a combination thereof. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS², can be performed by first selecting and isolating a precursor ion (MS¹), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications or other modifications, for example sequence variants. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications or sequence variants, or identifying post-translational modifications or sequence variants, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, mass spectrometry can be performed under native conditions. As used herein, the term “native conditions” can include performing mass spectrometry under conditions that preserve non-covalent interactions in an analyte. For a detailed review on native MS, refer to the review: Elisabetta Boeri Erba & Carlo Petosa, The emerging role of native mass spectrometry in characterizing the structure and dynamics of macromolecular complexes, 24 PROTEIN SCIENCE 1176-1192 (2015).

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools.” Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com/proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

This disclosure provides a method for identifying an amino acid sequence of a digested peptide of a protein of interest. In some exemplary embodiments, the method comprises (a) combining a peptide digest having digested peptides of a protein of interest with at least one heavy peptide standard to form a mixture, wherein said at least one heavy peptide standard includes a heavy isotope at or near each peptide terminus and an amino acid sequence of said at least one heavy peptide standard is a predicted amino acid sequence of a digested peptide of said protein of interest; (b) subjecting said mixture to analysis using liquid chromatography-mass spectrometry; (c) comparing a retention time and/or at least one mass spectrum of said at least one heavy peptide standard to a retention time and/or at least one mass spectrum of said digested peptides; and (d) using the comparison of (c) to identify the amino acid sequence of a digested peptide of said protein of interest.

In some exemplary embodiments, the comparing step may comprise determining whether the retention time of the at least one heavy peptide standard aligns with a retention time of a digested peptide. The retention times may be considered to be aligned if they are exactly the same or about the same, for example, less than 1% different, less than 0.5% different, or less than 0.1% different. The retention times may be compared by comparing at least one chromatogram of the at least one heavy peptide standard to at least one chromatogram of a digested peptide. The retention times may be considered to be aligned if the main peaks of the chromatograms are completely overlapping or substantially overlapping. Peaks may be considered substantially overlapping if, for example, the area of one peak is entirely or almost entirely within the area of another peak, or if most of the area of one peak is within the area of another peak, for example over 50%, over 60%, over 70%, over 80%, over 90%, over 95%, or over 99% of the peak area.

In some exemplary embodiments, an amount of peptide digest sample injected on the liquid chromatography column is about 100 fmol, about 200 fmol, about 300 fmol, about 400 fmol, about 500 fmol, about 600 fmol, about 700 fmol, about 800 fmol, about 900 fmol, about 1 pmol, about 2 pmol, about 3 pmol, about 4 pmol, about 5 pmol, about 6 pmol, about 7 pmol, about 8 pmol, about 9 pmol, about 10 pmol, or in a range between about 0.5 pmol and about 2 pmol. In some exemplary embodiments, an amount of heavy peptide standard applied to a liquid chromatography column is about 1 fmol, about 2 fmol, about 3 fmol, about 4 fmol, about 5 fmol, about 6 fmol, about 7 fmol, about 8 fmol, about 9 fmol, about 10 fmol, about 11 fmol, about 12 fmol, about 13 fmol, about 14 fmol, about 15 fmol, about 20 fmol, about 30 fmol, or between about 5 fmol and about 20 fmol.

This disclosure also provides a method for identifying a sequence variant of an antibody. In some exemplary embodiments, the method comprises (a) combining a peptide digest having digested peptides of an antibody with at least one heavy peptide standard to form a mixture, wherein said at least one heavy peptide standard includes a heavy isotope at or near each peptide terminus, and an amino acid sequence of said at least one heavy peptide standard is a predicted amino acid sequence of a digested peptide of said antibody featuring a sequence variant; (b) subjecting said mixture to analysis using liquid chromatography-mass spectrometry; (c) comparing a retention time and/or at least one mass spectrum of said at least one heavy peptide standard to a retention time and/or at least one mass spectrum of said digested peptides; and (d) using the comparison of (c) to identify a sequence variant of said antibody.

In some exemplary embodiments, the sequence variant may be a critical quality attribute.

In some exemplary embodiments, the antibody can be a bispecific antibody, a monoclonal antibody, a fusion protein, an antibody-drug conjugate, an antibody fragment, a biotherapeutic antibody, or an antibody pharmaceutical product.

In some exemplary embodiments, the chromatography step may comprise reversed phase liquid chromatography, ion exchange chromatography, size exclusion chromatography, affinity chromatography, hydrophobic interaction chromatography, hydrophilic interaction chromatography, mixed-mode chromatography, or a combination thereof.

In some exemplary embodiments, the mass spectrometer may be an electrospray ionization mass spectrometer, nano-electrospray ionization mass spectrometer, or an Orbitrap-based mass spectrometer, wherein said mass spectrometer is coupled to the liquid chromatography system.

In some exemplary embodiments, the at least one mass spectrum may be an MS¹ spectrum, an MS² spectrum (tandem mass spectrum), or an MS³ spectrum.

In some exemplary embodiments, the comparing step may comprise determining whether the retention time of the at least one heavy peptide standard aligns with a retention time of a digested peptide. The retention times may be considered to be aligned if they are exactly the same or about the same, for example, less than 1% different, less than 0.5% different, or less than 0.1% different. The retention times may be compared by comparing at least one chromatogram of the at least one heavy peptide standard to at least one chromatogram of a digested peptide. The retention times may be considered to be aligned if the main peaks of the chromatograms are completely overlapping or substantially overlapping. Peaks may be considered substantially overlapping if, for example, the area of one peak is entirely or almost entirely within the area of another peak, or if most of the area of one peak is within the area of another peak, for example over 50%, over 60%, over 70%, over 80%, over 90%, over 95%, or over 99% of the peak area.

In some exemplary embodiments, the comparing step comprises determining whether the MS¹ spectrum peaks of the at least one heavy peptide are shifted by the added mass of the heavy isotopes relative to a digested peptide. In some exemplary embodiments, the comparing step comprises determining whether the MS² spectrum peaks of the at least one heavy peptide are shifted by the added mass of one of the heavy isotopes relative to a digested peptide.

In some exemplary embodiments, a molar ratio of the peptide digest to the heavy peptide standard is between about 1:50 and 1:200, about 1:100, or 1:100.

In some exemplary embodiments, an amount of peptide digest sample injected on the liquid chromatography column is about 100 fmol, about 200 fmol, about 300 fmol, about 400 fmol, about 500 fmol, about 600 fmol, about 700 fmol, about 800 fmol, about 900 fmol, about 1 pmol, about 2 pmol, about 3 pmol, about 4 pmol, about 5 pmol, about 6 pmol, about 7 pmol, about 8 pmol, about 9 pmol, about 10 pmol, or in a range between about 0.5 pmol and about 2 pmol. In some exemplary embodiments, an amount of heavy peptide standard applied to a liquid chromatography column is about 1 fmol, about 2 fmol, about 3 fmol, about 4 fmol, about 5 fmol, about 6 fmol, about 7 fmol, about 8 fmol, about 9 fmol, about 10 fmol, about 11 fmol, about 12 fmol, about 13 fmol, about 14 fmol, about 15 fmol, about 20 fmol, about 30 fmol, or between about 5 fmol and about 20 fmol.

The method of the present invention may be applied to any protein of interest. In some exemplary embodiments, a particular application involves analysis of a protein of interest that is an antibody. In some exemplary embodiments, the protein of interest is a monoclonal antibody. In some exemplary embodiments, the protein of interest is a bispecific antibody. In some exemplary embodiments, the protein of interest is a recombinant protein. In some exemplary embodiments, the protein of interest is a fusion protein, for example a receptor fusion protein. In some exemplary embodiments, the protein of interest is a host cell protein.

It is understood that the present invention is not limited to any of the aforesaid protein(s), protein(s) of interest, antibody(s), protein alkylating agent(s), protein denaturing agent(s), protein reducing agent(s), digestive enzyme(s), sample(s), chromatographic method(s), mass spectrometer(s), database(s), bioinformatics tool(s), pH, temperature(s), or concentration(s), and any protein(s), protein(s) of interest, antibody(s), protein alkylating agent(s), protein denaturing agent(s), protein reducing agent(s), digestive enzyme(s), sample(s), chromatographic method(s), mass spectrometer(s), database(s), bioinformatics tool(s), pH, temperature(s), or concentration(s) can be selected by any suitable means.

The present invention will be more fully understood by reference to the following examples. They should not, however, be construed as limiting the scope of the invention.

### EXAMPLES

### Example 1. Heavy Peptide Standard Strategy

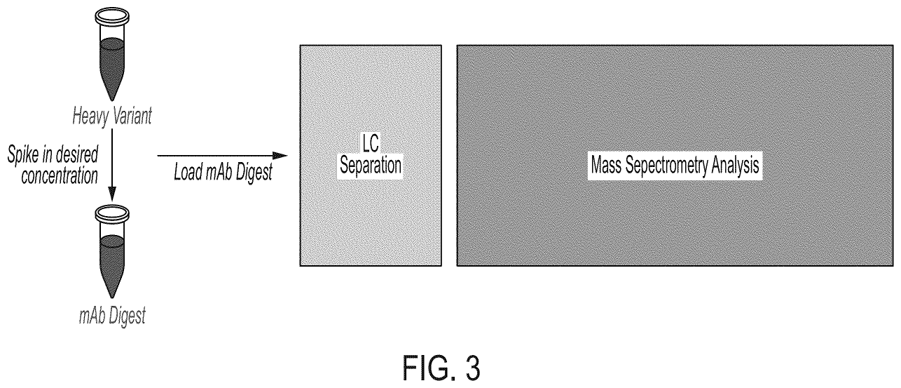

In order to confidently identify sequence variants of a protein of interest, a heavy peptide standard strategy was designed. An exemplary workflow is illustrated in FIG. 3. It should be understood that a variety of proteins of interest, liquid chromatography systems and mass spectrometry systems may be used in the method of the present invention.

Peptide standards having the amino acid sequence of known digested peptide fragments with sequence variants are synthesized with heavy isotopes at or near both peptide termini. The resulting heavy peptide standards will elute with substantially the same retention time as the corresponding sequence variant peptide fragment, allowing for one-to-one identification of a heavy peptide standard and sequence variant peptide. Mass spectrometry can then be used in order to differentiate the peptide standard and the sequence variant peptide, and to further confirm the exact amino acid sequence of the sequence variant peptide.

Placing heavy isotopes at or near both peptide termini allows for validation through retention time, MS¹ signal, and MS² fragmentation for more confident identification and sequence variant library compilation. Heavy peptide standards and the corresponding endogenous sequence variant peptides will always co-elute, negating run-to-run retention time variability. A novel feature of the method of the present invention is the use of heavy isotopes at or near both termini of a heavy peptide standard, which results in the production of heavy fragment ions using MS² analysis and allows for the convenient separation and identification of a heavy peptide standard and a corresponding sequence variant peptide. Composite MS² spectra allow for the simultaneous identification of light and heavy fragment ions in a spectrum. Using the method of the present invention, detailed determinations, like isoleucine versus leucine substitutions, and accurate pinpointing of sequence variant residues with multiple potential sites can be made.

The method of the present invention uses a small amount of sample (for example, about 1 pmol on column) and standard (for example, about 10 fmol on column) and can be used to validate sequence variants in historic samples. In some exemplary embodiments, variants may be separated and identified even with very short gradients, as low as 11 minutes, for example using Evosep One.

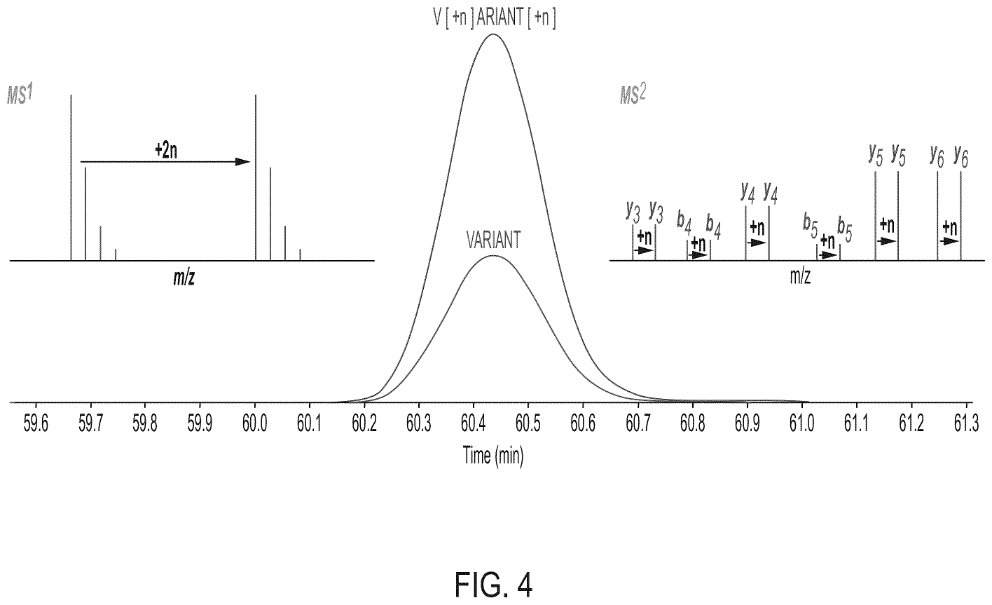

Co-elution and subsequent separation of a heavy peptide standard and a corresponding experimental peptide (featuring the same amino acid sequence) are illustrated in FIG. 4. A heavy peptide standard and a corresponding sequence variant peptide have aligned retention times, as indicated by substantially overlapping chromatographic peaks. In MS¹ analysis, the peaks of a heavy peptide standard and a corresponding sequence variant peptide are separated by the difference in mass attributable to the heavy isotopes. In MS² analysis, the peaks of the fragment ions (for example, a, b, c, x, y, or z ions) of a heavy peptide standard and a corresponding sequence variant peptide are separated by the difference in mass attributable to the heavy isotope of the respective peptide terminus.

### Example 2. NISTmAb Case Study

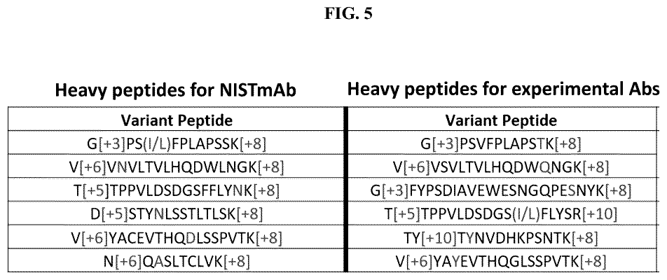

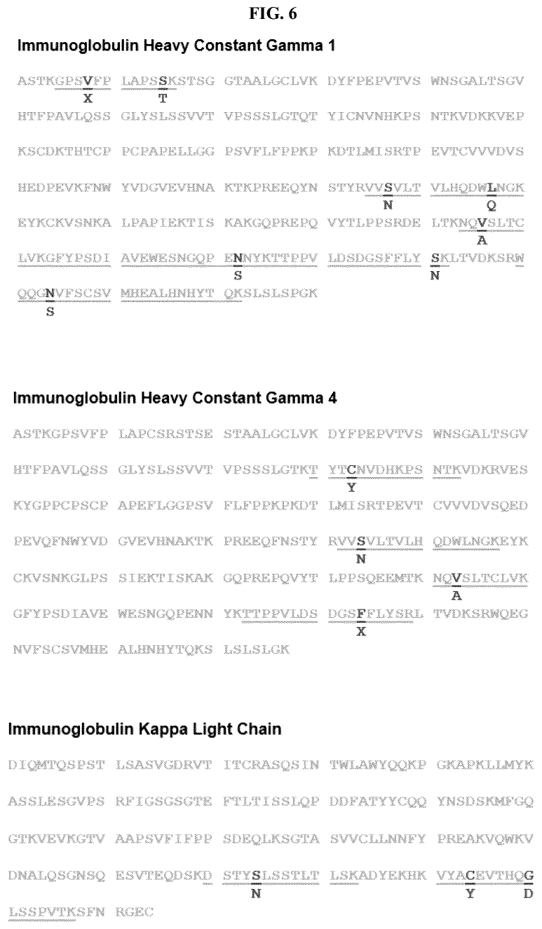

The method of the present invention was carried out using a NISTmAb standard antibody and experimental antibodies. Exemplary heavy peptide standards used for the NISTmAb standard and for experimental antibodies are shown in FIG. 5. Known sequence variants are highlighted in red. Additional mass attributable to heavy isotopes is indicated by blue numerals. Heavy peptide standard sequences were chosen to correspond to known sequence variants across heavy constant gamma and kappa light chains, as shown in FIG. 6.

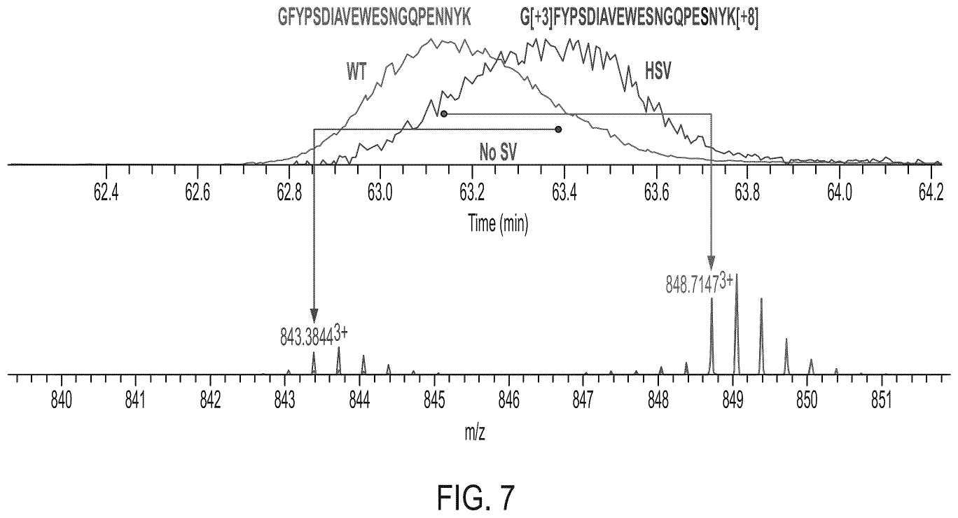

FIG. 7 shows a negative control using a NISTmAb sample. No sequence variant peptide is detected: only a wildtype peptide is detected from NISTmAb, which does not have the same retention time as the heavy peptide standard. In FIG. 7 and subsequent figures, the scale between extracted ion chromatograms (XICs) and mass spectra signal is not 1:1.

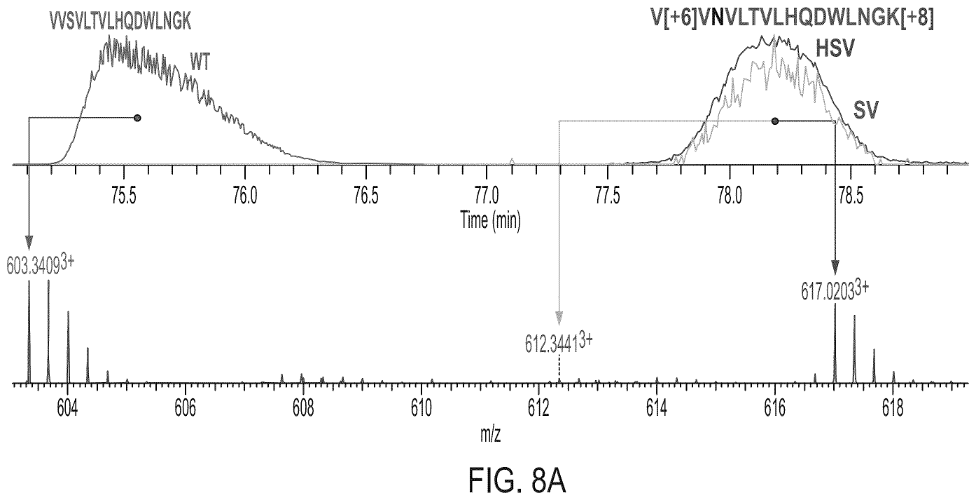

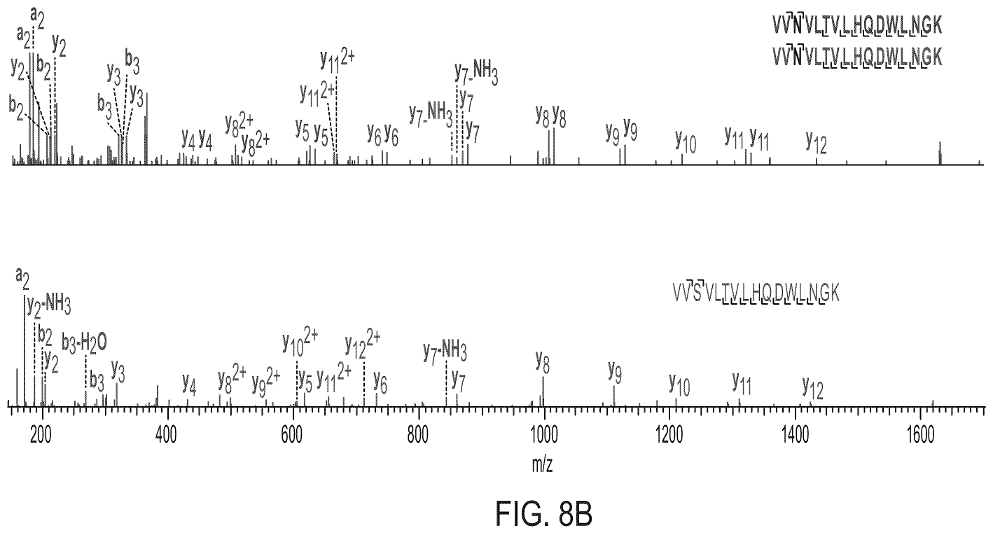

FIG. 8A shows the identification of a sequence variant peptide using the method of the present invention. The sequence variant peptide has a larger retention time than the corresponding wildtype peptide but the same retention time as the corresponding heavy peptide standard. The specific amino acid sequence is confirmed after MS² fragmentation, as shown in FIG. 8B.

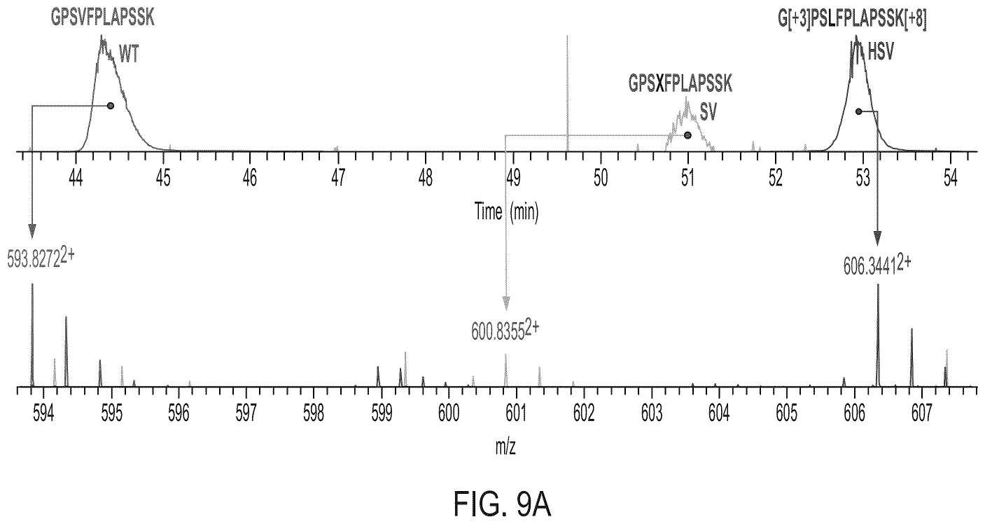

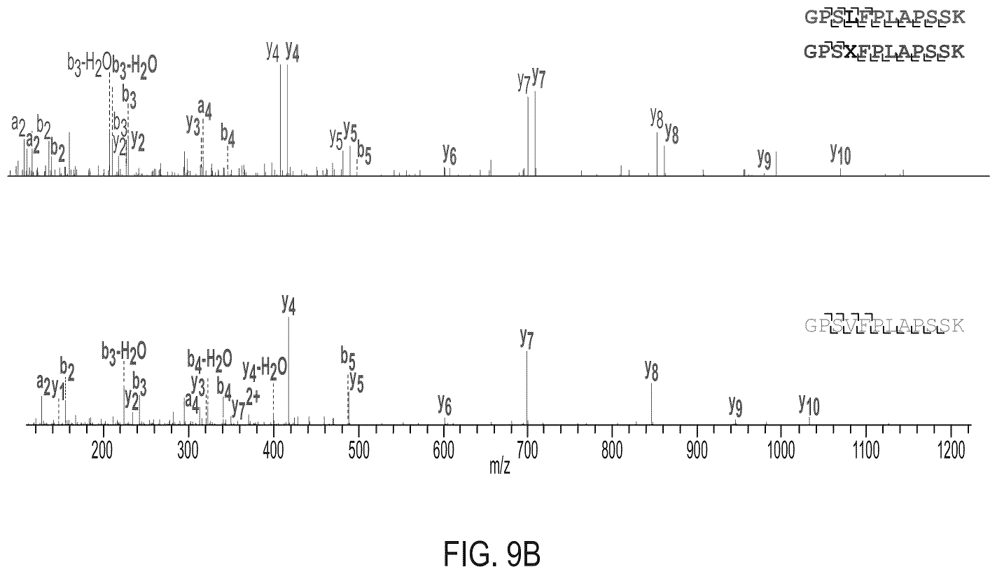

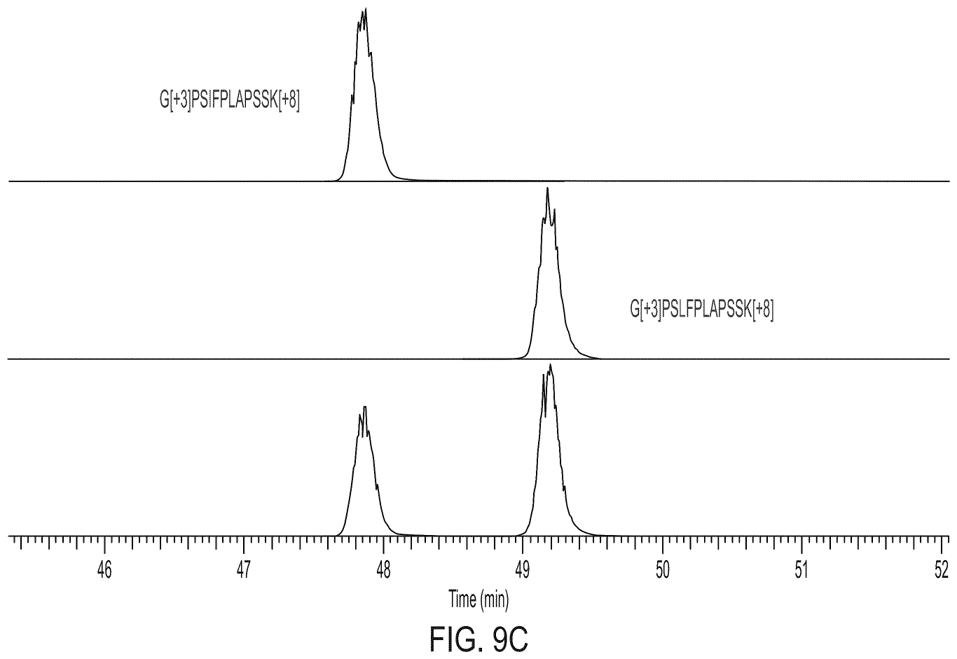

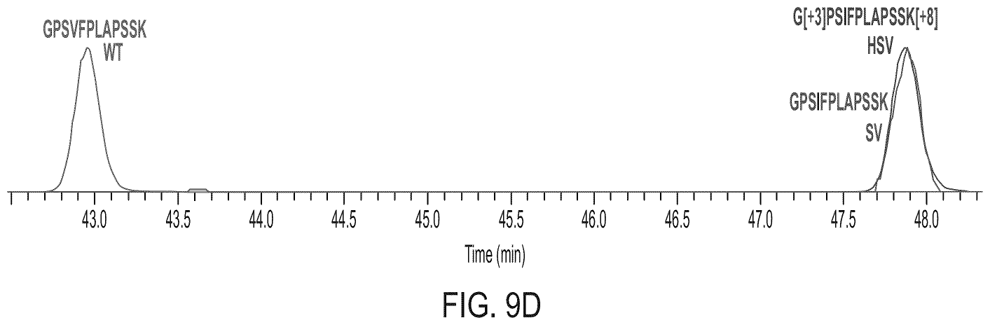

The method of the present invention can be used to positively distinguish between an amino acid sequence featuring a leucine or an isoleucine, which may otherwise be difficult due to their identical mass. FIG. 9A shows an example of a peptide where the retention time of the sequence variant peptide is smaller than the retention time of a heavy peptide standard representing a change to leucine. However, the MS² signal from the sequence variant peptide and heavy peptide standard match, indicating that the unknown amino acid may have the same mass as leucine, as shown in FIG. 9B. A comparison of heavy peptide standards representing a leucine variant compared to an isoleucine variant shows that the isoleucine variant is expected to have a smaller retention time, as shown in FIG. 9C. Directly comparing the heavy peptide standard representing an isoleucine variant to the sequence variant peptide confirms that the sequence variant features a change to an isoleucine, as shown in FIG. 9D.

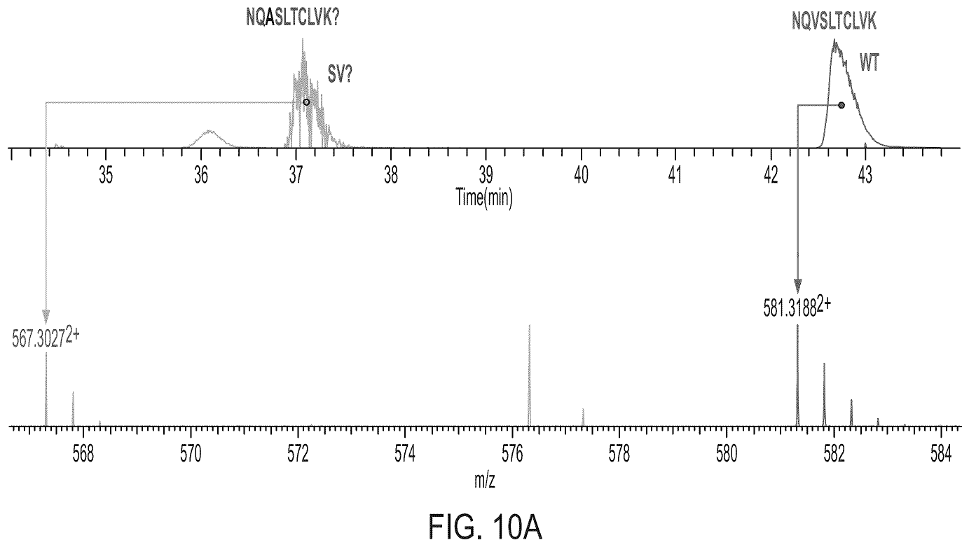

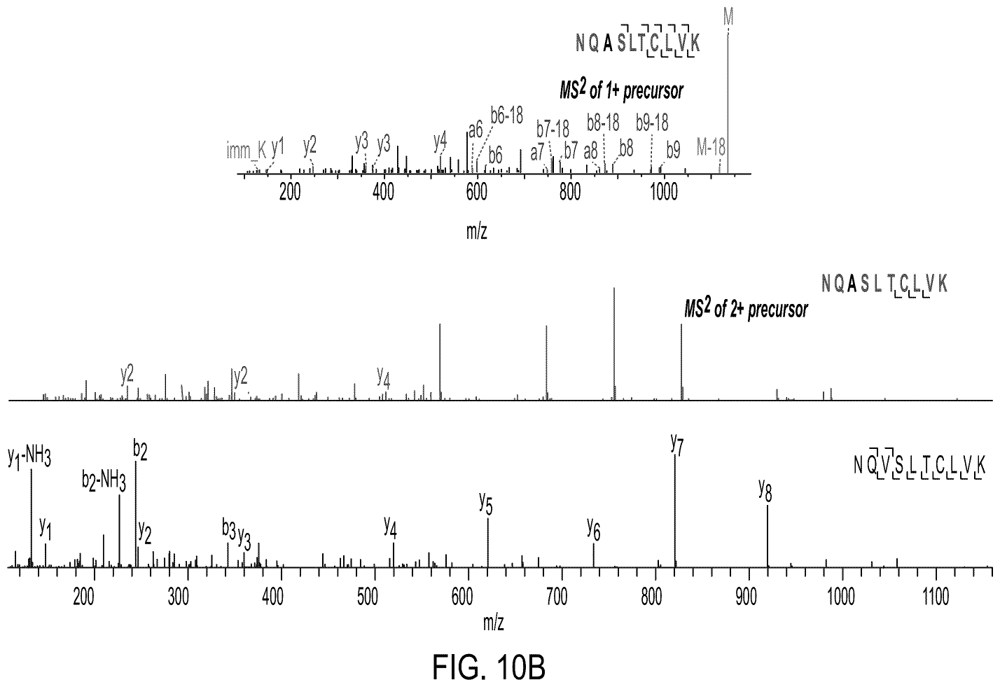

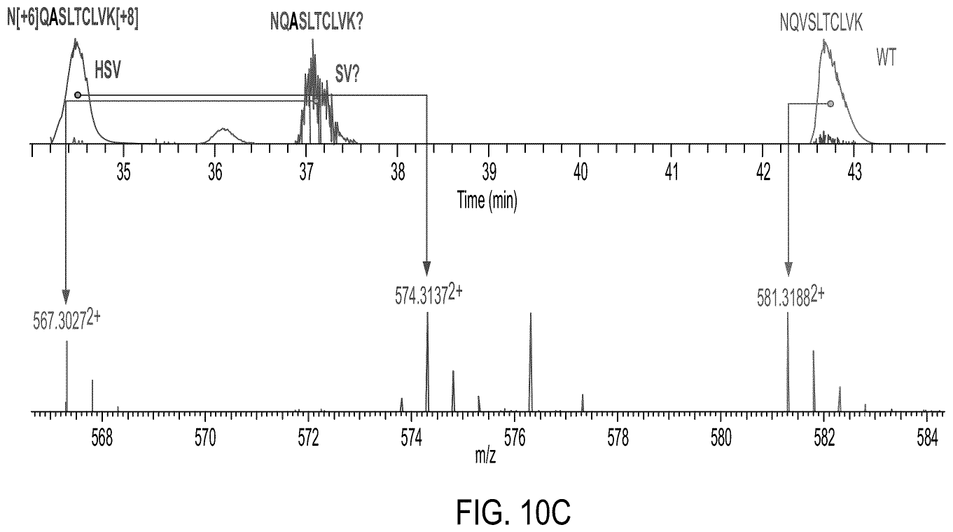

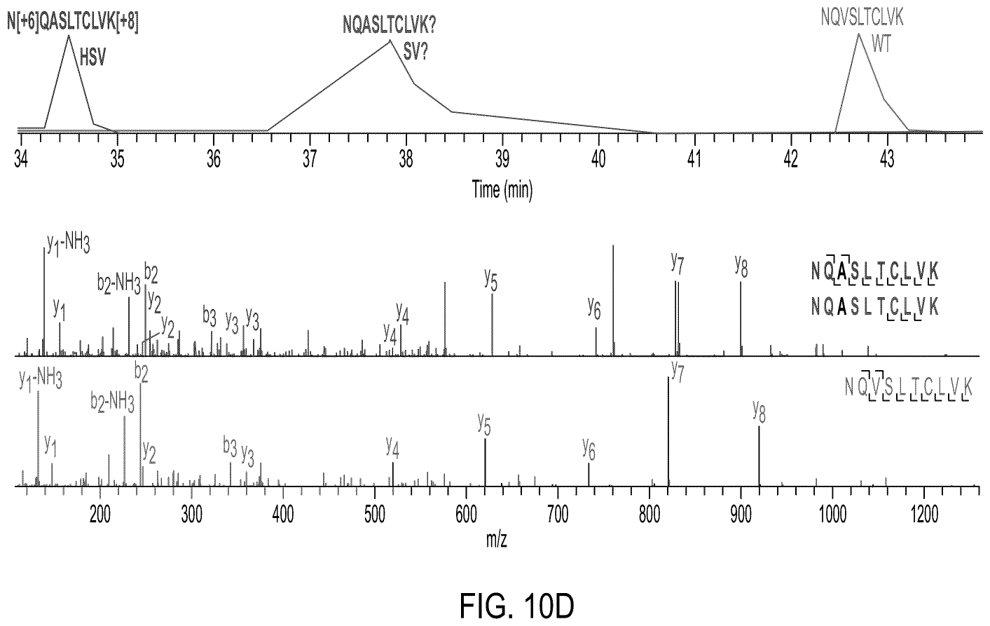

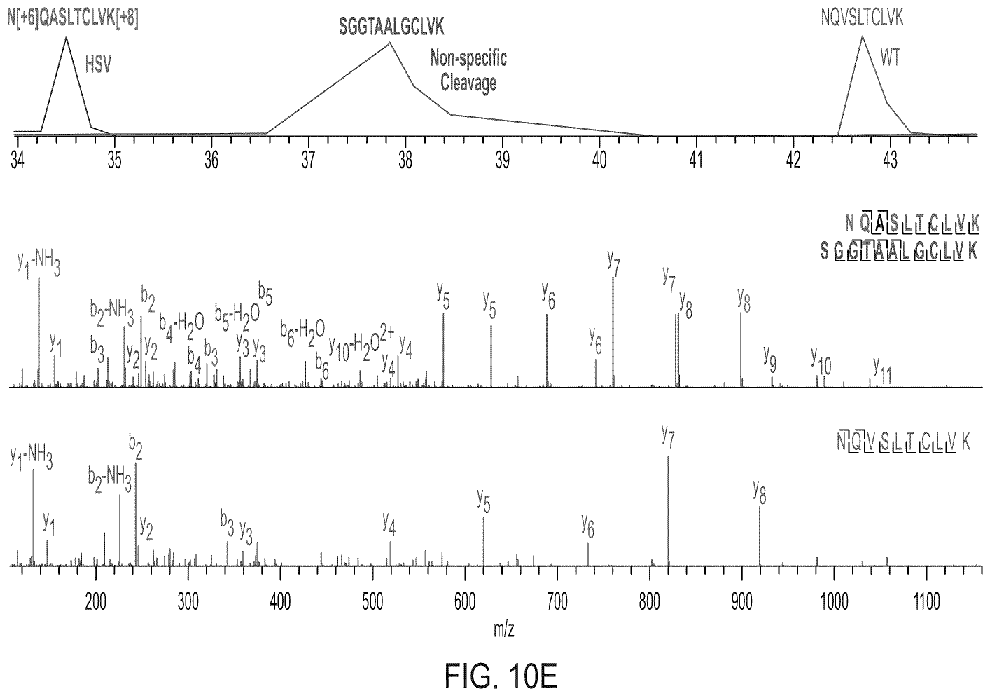

The method of the present invention can also be used to rule out false positive identifications of sequence variants. FIG. 10A shows LC and MS signal from a potential sequence variant, with a size corresponding to an alanine substitution. The MS² signal was insufficient for clearly identifying the amino acid sequence of this peptide, as shown in FIG. 10B. In order to confirm the identity of the sequence variant, a heavy peptide standard featuring the predicted amino acid sequence was used. The corresponding heavy peptide standard did not share the same retention time as the putative sequence variant peptide, as shown in FIG. 10C. MS² analysis in comparison to the heavy peptide standard revealed that the putative sequence variant was in fact a product of a non-specific cleavage, as shown in FIG. 10D and FIG. 10E. Using a heavy peptide standard to confirm or refute a putative sequence variant identification allows for confident identification compared to existing methods.

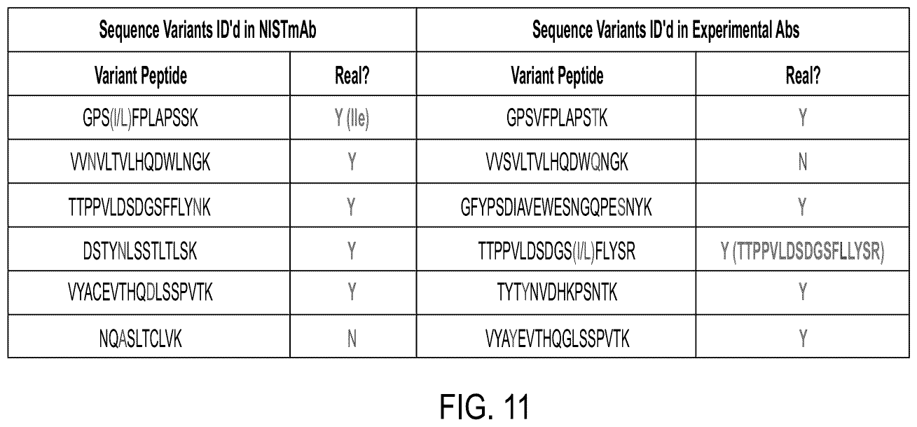

FIG. 11 shows a list of putative sequence variants identified from NISTmAb or from experimental antibodies, and whether the sequence variant identification was confirmed or refuted using the method of the present invention. Using the method of the present invention, it was possible to confirm true positive identifications, to rule out false positive identifications, and to identify the specific amino acid sequence of isoleucine/leucine variants.

### Example 3. Optimization of the Liquid Chromatography Gradient

The sample size of a sequence variant analysis may be very large, especially for design of experiments (DOE). In order to optimize the workflow of the method of the present invention, gradients of different speeds or durations were tested and compared for effectiveness in sequence variant identification.

An exemplary chromatography system useful with the present invention is Evosep One. Samples may be loaded onto an Evotip (a disposable trap column), and a gradient from two pumps elutes the sample peptides. During this elution, a secondary gradient from another two pumps offsets the first gradient to focus the sample peptides once introduced onto the analytical column. A high-pressure pump pushes the pre-formed gradients and pre-separated peptides through the analytical column.

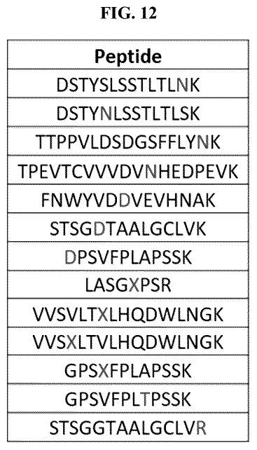

Five gradients were tested in total: an 11 (or 11.5) minute gradient, a 21 minute gradient, a 44 minute gradient and an 88 minute gradient using Evosep One, and a 95 minute gradient using ultra performance liquid chromatography (UPLC), for example Waters UPLC. Runs were benchmarked against high confidence sequence variants identified in a NISTmAb case study using 140 and 150 minute gradients, as shown in FIG. 12, as well as additional variants detected by data-dependent acquisition (DDA) at 5% false discovery rate (FDR) and manually inspected. NISTmAb sequence variants represent variants identified by both labs in Chapter 2 of State-of-the-Art and Emerging Technologies for Therapeutic Monoclonal Antibody Characterization Volume 2. In addition to sequence variant identification, gradients were compared for retention time reproducibility, quantification, and spectral quality.

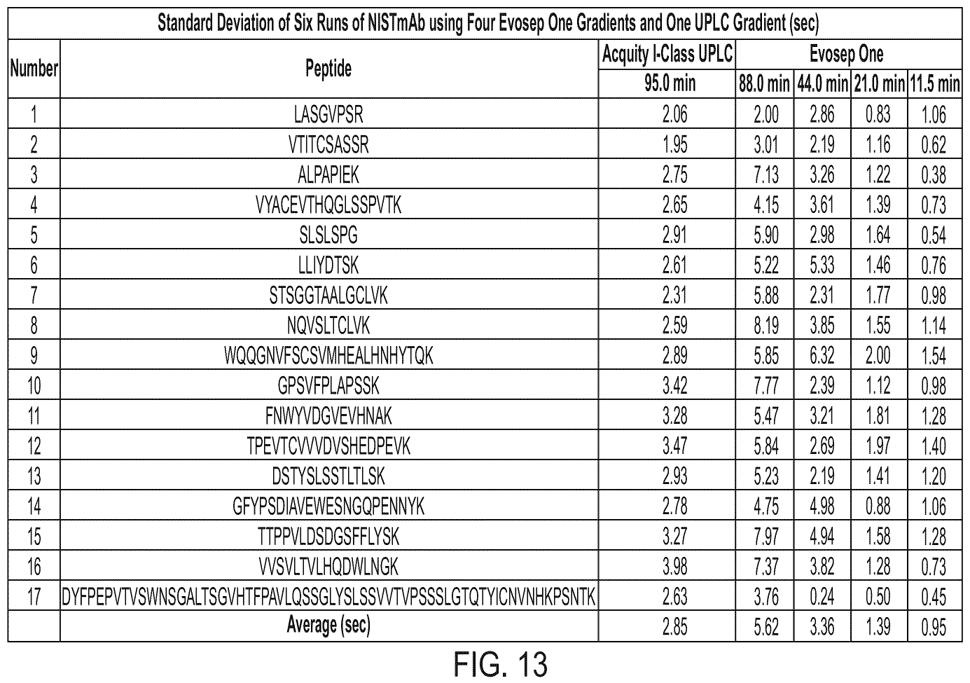

Standard deviation of retention time across the tested gradients and peptides is shown in FIG. 13. The retention time standard deviation for a 44 minute gradient using Evosep One is similar to a 95 minute UPLC run.

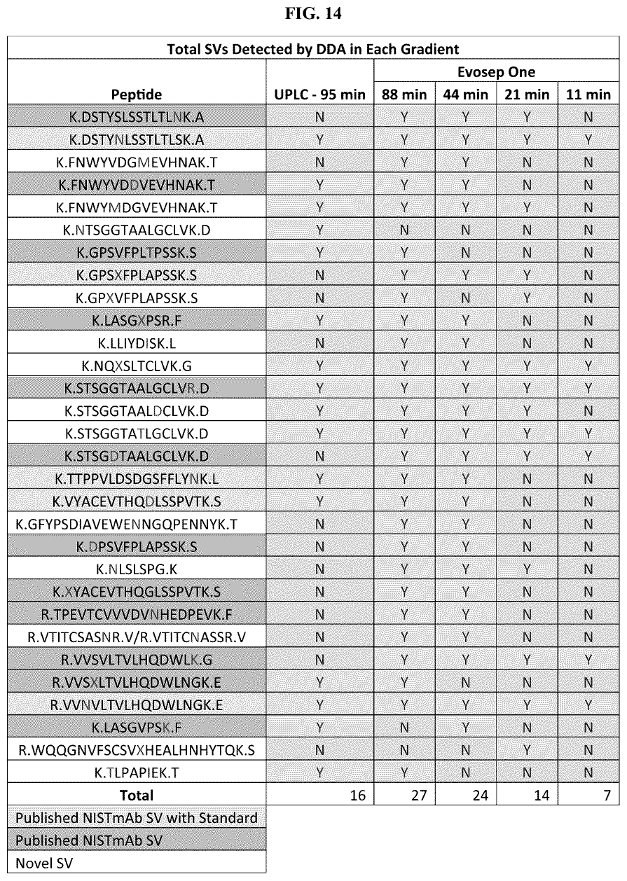

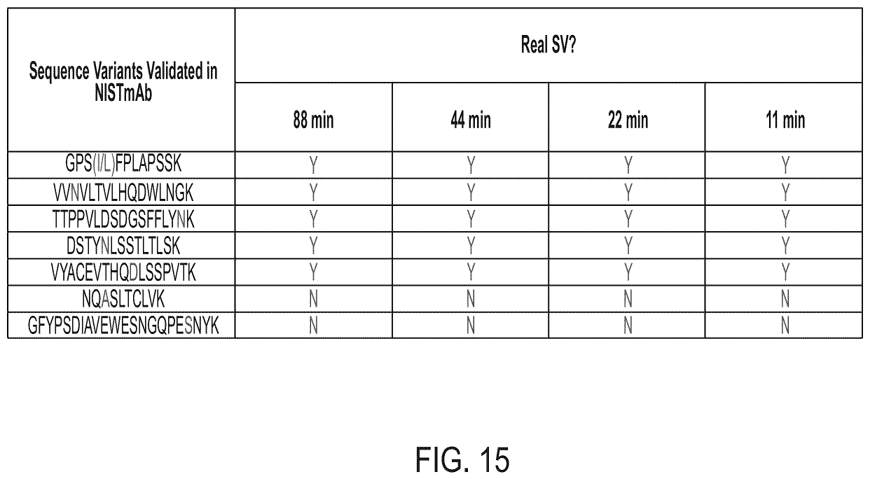

Sequence variant identification across the tested gradients and peptides is shown in FIG. 14. The 88 minute and 44 minute gradients using Evosep One identified more sequence variants than the 95 minute UPLC run, including many novel sequence variants. However, targeted MS² could be used to validate sequence variants even when using the 11 minute gradient, as shown in FIG. 15.

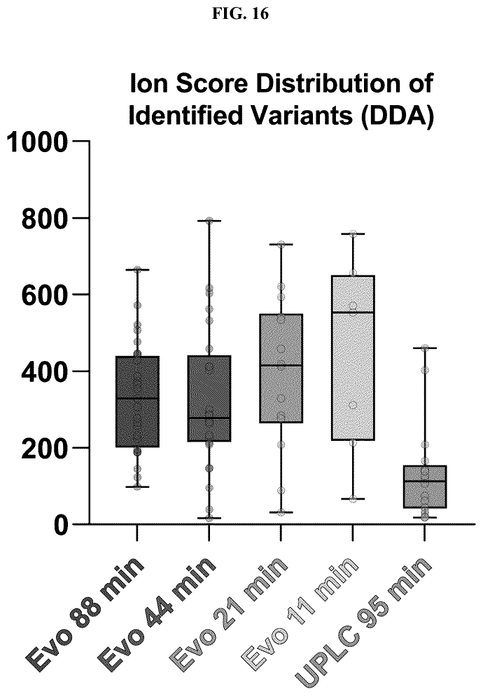

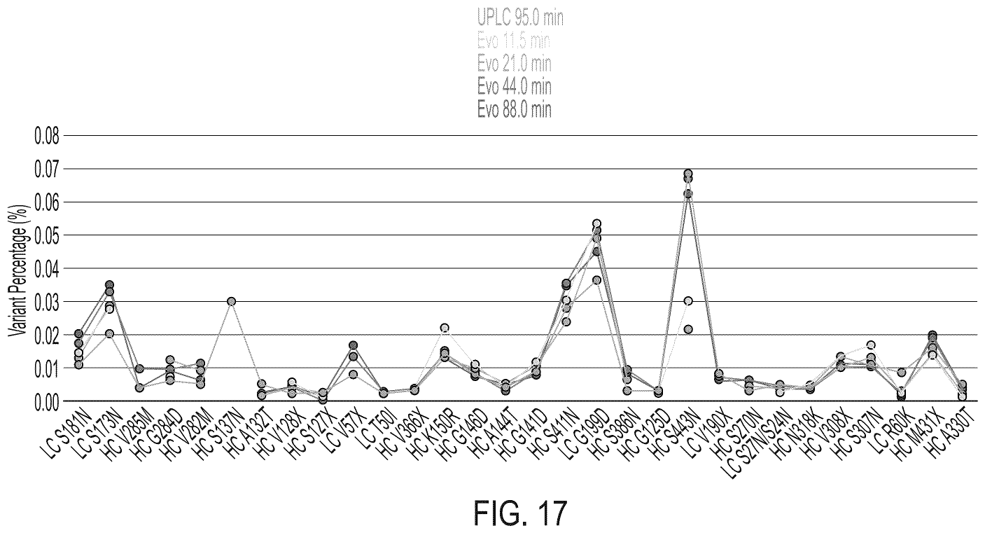

MS² spectral scores were higher for each of the Evosep One runs compared to the UPLC run, as shown in FIG. 16. A moderate overlap in quantitative values was observed among all of the tested gradients, as shown in FIG. 17. Peptides shown include extracted-ion chromatogram (XIC) extractions of sequence variants not identified by DDA.

As demonstrated above, the new heavy peptide standard method allows for simple and robust sequence variant validation for a protein of interest. This method reduces ambiguity in sequence variant analysis, increases confidence in results, and can be performed with relatively high throughput using short liquid chromatography gradients. The method of the present invention is not limited to sequence variant analysis but can be generally used to identify and/or confirm an amino acid sequence of any peptide, for example any digested peptide of any protein of interest.

What is claimed is:

1. A method for identifying an amino acid sequence of a digested peptide of a protein of interest, comprising:
(a) combining a peptide digest having digested peptides of a protein of interest with at least one heavy peptide standard to form a mixture, wherein said at least one heavy peptide standard includes a heavy isotope at or near each peptide terminus, and an amino acid sequence of said at least one heavy peptide standard is a predicted amino acid sequence of a digested peptide of said protein of interest;
(b) subjecting said mixture to analysis using liquid chromatography-mass spectrometry;
(c) comparing a retention time and/or at least one mass spectrum of said at least one heavy peptide standard to a retention time and/or at least one mass spectrum of said digested peptides; and
(d) using the comparison of (c) to identify the amino acid sequence of a digested peptide of said protein of interest.

2. The method of claim 1, wherein said amino acid sequence is a sequence variant.

3. The method of claim 2, wherein said sequence variant is a critical quality attribute.

4. The method of claim 1, wherein said protein of interest is an antibody, a bispecific antibody, a monoclonal antibody, a fusion protein, an antibody-drug conjugate, an antibody fragment, a host cell protein, or a protein pharmaceutical product.

5. The method of claim 1, wherein said chromatography step comprises reversed phase liquid chromatography, ion exchange chromatography, size exclusion chromatography, affinity chromatography, hydrophobic interaction chromatography, hydrophilic interaction chromatography, mixed-mode chromatography, or a combination thereof.

6. The method of claim 1, wherein said mass spectrometer is an electrospray ionization mass spectrometer, nano-electrospray ionization mass spectrometer, or an Orbitrap-based mass spectrometer, wherein said mass spectrometer is coupled to said liquid chromatography system.

7. The method of claim 1, wherein said comparing step comprises determining whether a retention time of said at least one heavy peptide standard aligns with a retention time of a digested peptide.

8. The method of claim 1, wherein said comparing step comprises determining whether MS¹ spectrum peaks of said at least one heavy peptide are shifted by the added mass of the heavy isotopes relative to MS¹ spectrum peaks of a digested peptide.

9. The method of claim 1, wherein said comparing step comprises determining whether MS² spectrum peaks of said at least one heavy peptide are shifted by the added mass of one of the heavy isotopes relative to MS² spectrum peaks of a digested peptide.

10. The method of claim 1, wherein a molar ratio of said peptide digest to said heavy peptide standard is between about 1:50 and about 1:200, or about 1:100.
